(19) United States
(12) Patent Application Publication
(10) Pub. No.: US 2009/0011488 A1
(43) Pub. Date: Jan. 8, 2009

(54) METHODS FOR STORING COMPOSITIONS USEFUL FOR SYNTHESIZING NUCLEIC ACID MOLECULES

(75) Inventor: Mingjie Zhou, Kirkland, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: ROSETTA INPHARMATICS, LLC, Seattle, WA (US)

(21) Appl. No.: 12/210,066

(22) Filed: Sep. 12, 2008

Related U.S. Application Data
(63) Continuation of application No. 10/867,376, filed on Jun. 14, 2004, now abandoned.
(60) Provisional application No. 60/495,977, filed on Aug. 18, 2003.

(51) Int. Cl. C12N 9/00

(52) U.S. Cl. 435/183

(57) ABSTRACT

In one aspect, the present invention provides methods for storing a composition useful for synthesizing nucleic acid molecules. The methods of this aspect of the invention include the steps of: (a) freezing multiple aliquots of a liquid composition comprising from 1000 units/mL to 5000 units/mL of a reverse transcriptase, or from 10,000 units/mL to 50,000 units/mL of an RNA polymerase, wherein the multiple aliquots of the liquid composition are disposed within multiple receptacles defined by a container body; and (b) a step selected from the group consisting of (1) storing the frozen aliquots at a temperature below −15° C., and (2) drying the frozen aliquots to produce dried aliquots of the composition, wherein each dried aliquot of the composition comprises an amount of water that is less than 0.1% by weight of the dried aliquot, and storing the dried aliquots at a temperature below −15° C.

### CROSS-REFERENCES TO RELATED APPLICATIONS

This application is a continuation of application Ser. No. 10/867,376, filed Jun. 14, 2004, which claims the benefit of Provisional Application No. 60/495,977, filed Aug. 18, 2003, the disclosures of which are incorporated by reference herein.

### BACKGROUND

The present invention relates to the storage of reaction mixtures useful for synthesizing nucleic acid molecules, to reaction mixtures useful for synthesizing nucleic acid molecules, and to containers that include reaction mixtures useful for synthesizing nucleic acid molecules.

Many techniques for analyzing the expression of gene populations require the synthesis of nucleic acid molecules which may be used as hybridization probes, or as intermediates in the synthesis of hybridization probes. The synthesis of these nucleic acid molecules may require several steps which may each require a different reaction mixture.

For example, published international patent application PCT/US02/22787 (published on Mar. 13, 2003, under International Publication Number WO 03/020979 A1) discloses methods for preparing nucleic acid molecules useful for screening DNA arrays. The disclosed methods include the steps of: (a) using an RNA template to enzymatically synthesize a first DNA molecule that is complementary to at least 50 contiguous bases of the RNA template; (b) using the first DNA molecule as a template to enzymatically synthesize a second DNA molecule, thereby forming a double-stranded DNA molecule wherein the first DNA molecule is hybridized to the second DNA molecule; (c) using the first or second DNA molecule of the double-stranded DNA molecule as a template to enzymatically synthesize a first RNA molecule that is complementary to either the first DNA molecule or to the second DNA molecule; and (d) using the first RNA molecule as a template to enzymatically synthesize a third DNA molecule that is complementary to the first RNA molecule. The third DNA molecule may be labelled and used to screen DNA microarrays comprising numerous target molecules. The foregoing methods include four distinct nucleic acid synthetic reactions.

Further, there is an increasing trend toward automation of techniques for simultaneously analyzing the expression of gene populations in numerous samples. Thus, for example, a single automated system can extract RNA from numerous samples of living tissue (e.g., from hundreds of samples of living tissue), synthesize and label nucleic acid molecules (e.g., cDNA or cRNA) using the extracted RNA as a template, simultaneously perform numerous hybridization reactions using the labelled nucleic acid molecules as probes (e.g., use the labelled nucleic acid molecules as probes to screen gene chips), and measure and record the results of the hybridization experiments.

It is time-consuming to prepare fresh reaction mixtures for each nucleic acid synthetic reaction, or group of reactions. Moreover, inadvertent variation in the amounts of components included in each batch of reaction mixture, and variation in the quality of different batches of individual components included in each batch of reaction mixture, can affect the reproducibility of the synthetic reaction, and the quality of the nucleic acid products. These problems are especially significant when numerous samples (perhaps hundreds or thousands of samples) are processed.

Consequently, there is a need for methods for storing nucleic acid synthesis reaction mixtures, including the enzyme(s) that catalyze the nucleic acid synthetic reaction(s), so that a single batch of reaction mixture, sufficient for numerous synthetic reactions, can be stored until used. Preferably, the storage method permits a batch of nucleic acid reaction mixture to be divided into numerous aliquots, that are each sufficient for at least one nucleic acid synthetic reaction, that can be stored until they are used. There is also a need for reaction mixtures that can be stored until they are used for synthesizing nucleic acid molecules.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect the present invention provides methods for storing a composition useful for synthesizing nucleic acid molecules. The methods of this aspect of the invention include the steps of: (a) freezing multiple aliquots of a liquid composition, wherein each aliquot comprises from 1000 units/mL to 5000 units/mL of a reverse transcriptase, or from 10,000 units/mL to 50,000 units/mL of an RNA polymerase, wherein the multiple aliquots of the liquid composition are disposed within multiple receptacles defined by a container body; and (b) a step selected from the group consisting of (1) storing the frozen aliquots at a temperature below −15° C., and (2) drying the frozen aliquots to produce dried aliquots of the composition, wherein each dried aliquot of the composition comprises an amount of water that is less than 0.1% by weight of the dried aliquot, and storing the dried aliquots at a temperature below −15° C.

In another aspect, the present invention provides containers that each include multiple receptacles, wherein at least 50% of the receptacles contain a composition selected from the group consisting of (a) a frozen liquid composition comprising from 1000 units/mL to 5000 units/mL of a reverse transcriptase, (b) a frozen liquid composition comprising from 10,000 units/mL to 50,000 units/mL of an RNA polymerase, and (c) a non-liquid composition comprising a reverse transcriptase or an RNA polymerase and an amount of water that is less than 0.1%, by weight, of the non-liquid composition.

In another aspect, the present invention provides compositions useful for synthesizing nucleic acid molecules. A first group of compositions of this aspect of the invention each include: a reverse transcriptase, a Tris buffer, a potassium salt, a magnesium salt, two different nucleotides, an oligonucleotide, a reducing agent, and an amount of water that is less than 0.1%, by weight, of the composition.

A second group of compositions of this aspect of the invention each include: an RNA polymerase, a Tris buffer, a sodium salt, a magnesium salt, two different nucleotides, a reducing agent, and an amount of water that is less than 0.1%, by weight, of the composition.

The methods and containers of the invention are useful for storing reaction mixtures, including a reverse transcriptase or an RNA polymerase, useful for synthesizing nucleic acid molecules. In particular, the methods and containers of the invention are useful for storing multiple, separate, aliquots of reaction mixtures, including a reverse transcriptase or an RNA polymerase, useful for synthesizing nucleic acid molecules.

For example, many modern techniques for analyzing gene expression patterns in living organisms include the step of synthesizing DNA and/or RNA molecules from numerous samples obtained from living organisms (e.g., nucleic acid samples obtained from living organisms treated with different drugs), and either analyze the identity and/or amount of the synthesized DNA or RNA molecules, or subject the synthesized DNA or RNA molecules to subsequent processing. The methods and containers of the present invention can be used to store numerous, identical, reaction mixtures, which can subsequently be thawed, or rehydrated, and used. Thus, a person synthesizing RNA or DNA from numerous samples does not have to make numerous reaction mixtures immediately before beginning the synthetic reactions. The methods and containers of the present invention thereby facilitate use of a high-throughput system for simultaneously, or sequentially, synthesizing RNA or DNA from numerous samples, and optionally using the synthesized nucleic acid molecules to perform numerous hybridization reactions.

For example, the methods and containers of the invention facilitate the use of the Fully Automated System (abbreviated as FAS system), for simultaneously synthesizing numerous nucleic acid probes, and using the probes to perform numerous hybridization reactions (e.g., screen numerous gene chips), which is disclosed in U.S. provisional patent application Ser. No. 60/432,200, filed Dec. 10, 2002 (which application is incorporated herein by reference).

The compositions of the invention are useful, for example, for synthesizing nucleic acid molecules. In particular, the compositions of the invention are useful, for example, in nucleic acid synthesis protocols and systems in which the gene expression profile of numerous samples is measured. The compositions of the invention can be included in a container of the invention, and/or used in the practice of the methods of the invention.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a perspective view of a representative container of the present invention that includes multiple receptacles.

FIG. 2 shows a portion of a transverse cross-section of the representative container shown in FIG. 1.

### DETAILED DESCRIPTION

Unless specifically defined herein, all terms used herein have the same meaning as they would to one skilled in the art of the present invention. Practitioners are particularly directed to Sambrook et al. (1989) Molecular Cloning: A Laboratory Manual, 2^{nd} ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

In one aspect, the present invention provides methods for storing a composition useful for synthesizing nucleic acid molecules. The methods of this aspect of the invention include the steps of: (a) freezing multiple aliquots of a liquid composition, wherein each aliquot comprises from 1000 units/mL to 5000 units/mL of a reverse transcriptase, or from 10,000 units/mL to 50,000 units/mL of an RNA polymerase, wherein the multiple aliquots of the liquid composition are disposed within multiple receptacles defined by a container body; and (b) a step selected from the group consisting of (1) storing the frozen aliquots at a temperature below −15° C., and (2) drying the frozen aliquots to produce dried aliquots of the composition, wherein each dried aliquot of the composition comprises an amount of water that is less than 0.1% by weight of the dried aliquot, and storing the dried aliquots at a temperature below minus 15° C. (abbreviated as −15° C.). The abbreviation mL means milliliter.

With respect to the units of reverse transcriptase activity, one unit of reverse transcriptase catalyzes the incorporation of 1 nmole of dTTP into acid-insoluble material in 10 minutes at 37° C. in 50 mM Tris-HCl (pH 8.3), 75 mM KCl, 1 mM MnSO₄, 1 mM DTT, and 0.5 mM dTTP, using 200 μM oligo(dT) 12-18-primed poly(A)n as template.

With respect to the units of RNA polymerase activity, one unit of RNA polymerase catalyzes the incorporation of 1 nmole of a labeled ribonucleoside triphosphate into RNA in 1 hour at 37° C. in 40 mM Tris-HCl (pH 7.5), 6 mM MgCl₂, 10 mM NaCl, 2 mM spermidine, 10 mM DTT, and 0.5 mM each NTP using a DNA template with the appropriate T7 promoter.

With respect to the units of inorganic pyrophosphatase activity, one unit of inorganic pyrophosphatase is the amount of enzyme that generates 40 nmols of phosphate per minute from pyrophosphate during a 10 minute reaction at 75° C. in 50 mM Tricine (pH 8.5), 1 mM MgCl₂, 0.32 mM pyrophosphate, in a reaction volume of 0.5 ml.

Liquid compositions that include a reverse transcriptase: Any reverse transcriptase (abbreviated as RT) can be included in the liquid compositions, such as the RTs derived from Moloney murine leukemia virus (abbreviated as MMLV-RT, such as the MMLV-RT sold under the trade name M4410K by Epicenter, Madison Wis.), avian myeloblastosis virus (abbreviated as AMV-RT), bovine leukemia virus (abbreviated as BLV-RT), Rous sarcoma virus (abbreviated as RSV) and human immunodeficiency virus (abbreviated as HIV-RT). Some reverse transcriptases possess RNase H activity (RNase H⁺ reverse transcriptases), while other reverse transcriptases lack RNase H activity (RNase H⁻ reverse transcriptases). An example of a reverse transcriptase lacking RNaseH activity is the reverse transcriptase sold under the trade name SUPERSCRIPT II™ by Invitrogen Life Technologies, Carlsbad, Calif.

The reverse transcriptase is present in the liquid composition at a concentration in the range of from 500 units/mL to 10,000 units/mL, such as from 1000 units/mL to 5000 units/mL.

In embodiments of the invention in which the liquid composition includes a reverse transcriptase, the liquid composition also typically includes components required for one or more chemical reactions catalyzed by the reverse transcriptase. Thus, the liquid composition may contain components that permit, or promote, the synthesis of a DNA molecule that has a nucleotide sequence that is complementary to the nucleotide sequence of a substrate RNA molecule, wherein the synthesis of the DNA molecule is catalyzed by the reverse transcriptase. For example, the liquid composition can include a Tris buffer, a potassium salt (e.g., potassium chloride), a magnesium salt (e.g., magnesium chloride), at least two different nucleotides, an oligonucleotide (typically a population of oligonucleotide molecules), and a reducing agent (e.g., dithiothreitol). The liquid composition can optionally include an RNase inhibitor, such as RNAguard™, sold by Amersham, or such as RNasin® sold by Promega (2800 Woods Hollow Road, Madison Wis. 53711, USA).

Tris is the abbreviation for Tris-(hydroxymethyl)aminomethane which may be used as a free base, but is more typically used as a salt, such as a hydrochloride salt (Tris-HCL).

The liquid compositions may include two, three, four, or more, different nucleotides. For example, the liquid compositions may include two, three or all four of the following nucleotides: adenine, cytosine, guanine, and thymidine. The liquid compositions may also include nucleotide derivatives wherein one or more of the base moiety, sugar moiety and/or phosphate backbone is/are modified. For example, a nucleotide may include one of the following modified base moieties: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, and 2,6-diaminopurine. Again by way of example, a nucleotide may include one of the following modified sugar moieties: arabinose, 2-fluoroarabinose, xylulose, and hexose. Again by way of example, a nucleotide may include one of the following modified phosphate backbones: a phosphorothioate, a phosphorodithioate, a phosphoramidothioate, a phosphoramidate, a phosphordiamidate, a methylphosphonate, an alkyl phosphotriester, and a formacetal. As used herein, the term “nucleotide” also encompasses substituted organic bases useful for making PNA (e.g., N-(2-aminoethyl)glycine having an organic base attached to the glycine nitrogen via a methylene carbonyl linker, such as described, for example, by Nielsen, P. et al., Bioconjug. Chem. 5:3-7 (1994)). PNA is a DNA mimic having a peptide-like, inorganic backbone.

The oligonucleotide molecules prime synthesis of DNA molecules that each has a nucleotide sequence that is complementary to the sequence of an RNA template. The oligonucleotides are typically DNA oligonucleotides in the range of ten to 60 bases in length. Oligonucleotides that are useful for priming the synthesis of the DNA molecules can hybridize to any portion of the RNA template molecules, including the poly A tail. All of the oligonucleotide molecules may have the same nucleic acid sequence, or there may be two, or more, different populations of oligonucleotides having different nucleic acid sequences.

In some embodiments of the methods of the invention, the liquid composition includes: (a) a reverse transcriptase that is present in the liquid composition at a concentration in the range of from 1000 units/mL to 5000 units/mL; (b) a Tris buffer that is present in the liquid composition at a concentration in the range of from 10 mM to 100 mM; (c) a potassium salt that is present in the liquid composition at a concentration in the range of from 10 mM to 100 mM; (d) a magnesium salt that is present in the liquid composition at a concentration in the range of from 1.0 mM to 10 mM; (e) nucleotides that are each present in the liquid composition at a concentration in the range of from 0.5 mM to 5 mM; (f) an oligonucleotide that is present in the liquid composition at a concentration in the range of from 0.01 mM to 0.05 mM; and (g) a reducing agent that is present in the liquid composition at a concentration in the range of from 2.0 mM to 20 mM. A specific representative example of a liquid composition that includes a reverse transcriptase is set forth in Table 1 herein.

Liquid compositions that include an RNA polymerase: Examples of RNA polymerases that can be included in the liquid composition are a T7 RNA polymerase, such as the T7 RNA polymerase sold under the trade name TU950K by Epicenter, Madison Wis., and, for example, cloned T7 RNA Polymerase sold by Ambion, 2130 Woodward, Austin, Tex. 78744, USA. The RNA polymerase is present in the liquid composition at a concentration in the range of from 10,000 units/mL to 50,000 units/mL.

In embodiments of the invention in which the liquid composition includes an RNA polymerase, the liquid composition also typically includes components required for one or more chemical reactions catalyzed by the RNA polymerase. Thus, the liquid composition may contain components that permit, or promote, the synthesis of RNA molecules that are complementary to a template DNA molecule, wherein the synthesis of the RNA molecules is catalyzed by the RNA polymerase. For example, the liquid composition can include a Tris buffer, a sodium salt (e.g., sodium chloride), a magnesium salt (e.g., magnesium chloride), at least two different nucleotides, and a reducing agent (e.g., dithiothreitol). The liquid composition can optionally include an RNase inhibitor, such as RNAguard™, spermidine and/or a pyrophosphatase, abbreviated as IPPase, (i.e., a soluble inorganic pyrophosphatase which hydrolyzes inorganic pyrophosphate (PPᵢ) to inorganic phosphate (Pᵢ)).

The liquid composition may include two, three, four, or more, different nucleotides. For example, the liquid composition may include two, three or all four of the following nucleotides: adenine, cytosine, guanine, and uracil. The liquid composition may also include any of the following nucleotide derivatives: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, 13-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, and 2,6-diaminopurine. Again by way of example, a nucleotide may include one of the following modified sugar moieties: arabinose, 2-fluoroarabinose, xylulose, and hexose. Again by way of example, a nucleotide may include one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, and substituted organic bases useful for making PNA (e.g., N-(2-aminoethyl)glycine having an organic base attached to the glycine nitrogen via a methylene carbonyl linker, such as described, for example, by Nielsen, P. et al., Bioconjug. Chem. 5:3-7 (1994)).

The liquid composition may also include an amino allyl derivatized uracil nucleotide. The amino allyl derivatized uracil nucleotide can be coupled, for example, via an aminoallyl linkage, to N-hydroxysuccinimide ester derivatives (NHS derivatives) of dyes (e.g., Cy-NHS, Cy3-NHS and/or Cy5-NHS).

In some embodiments of the methods of the invention, the liquid composition includes: (a) an RNA polymerase that is present in the liquid composition at a concentration in the range of from 10 units/μL to 50 units/μL; (b) a Tris buffer that is present in the liquid composition at a concentration in the range of from 10 mM to 100 mM; (c) a sodium salt that is present in the liquid composition at a concentration in the range of from 2.0 mM to 20 mM; (d) a magnesium salt that is present in the liquid composition at a concentration in the range of from 1.0 mM to 15 mM; (e) adenine, cytosine and guanine that are each present in the liquid composition at a concentration in the range of from 0.5 mM to 5.0 mM; (f) uracil that is present in the liquid composition at a concentration in the range of from 0.2 mM to 2.0 mM; (g) an amino allyl uracil nucleotide that is present in the liquid composition at a concentration in the range of from 0.05 mM to 1.0 mM; and (h) a reducing agent that is present in the liquid composition at a concentration in the range of from 2.0 mM to 20 mM. In these embodiments, the liquid composition may also include spermidine that is present in the liquid composition at a concentration in the range of from 100 units/mL to 500 units/mL. The liquid composition may also include an RNase inhibitor that is present in the liquid composition at a concentration in the range of from 100 units/mL to 500 units/mL. The liquid composition may also include an IPPase that is present in the liquid composition at a concentration in the range of from 10 units/mL to 50 units/mL. A specific representative example of a liquid composition that includes an RNA polymerase is set forth in Table 2 herein.

Containers useful for storing multiple aliquots of the liquid compositions: multiple aliquots of the liquid compositions are disposed within multiple receptacles defined by a container body. Useful containers can withstand freezing and thawing at least once, and include multiple receptacles (e.g., wells) for receiving and retaining aliquots (e.g., aliquots that each has a volume of from 1 μl to 100 μl) of liquid composition. For example, the container can include between 10 and 100 receptacles. Examples of useful containers for storing multiple aliquots of the liquid compositions include 96-well plastic plates manufactured by Island Scientific (7869 NE Day Rd West, Bainbridge Island, Wash. 98110), or by MWG Biotech (4170 Mendenhall Oaks Parkway, Suite 160, High Point, N.C. 27265).

Storing aliquots of the liquid compositions: In some embodiments of the methods of the invention, frozen aliquots of the liquid composition are stored at a temperature below minus 15° C. (−15° C.). Aliquots of the liquid composition can be frozen by any means, such as by placing the container containing the aliquots of the liquid composition into a freezer where the container is incubated at a temperature below the freezing point of the liquid composition until the aliquots of the liquid composition freeze. It is usually desirable to freeze the aliquots of the liquid composition as quickly as possible in order to preserve the biological activity of the enzyme.

The frozen aliquots can be stored in a freezer at a temperature below −15° C. For example, the frozen aliquots can be stored in a freezer at a temperature between −15° C. and −90° C. The frozen aliquots can be stored for a desired time period, such as for at least 24 hours, or for at least one week, or for between one week and one month.

In some embodiments of the methods of the invention, frozen aliquots of the liquid composition are dried to produce dried aliquots of the composition, wherein each dried aliquot of the composition comprises an amount of water that is less than 0.1% by weight of the dried aliquot, and the dried aliquots are stored at a temperature below −15° C. In some embodiments of the methods of the invention, each dried aliquot of the composition comprises an amount of water that is less than 0.05% by weight of the dried aliquot. In some embodiments of the methods of the invention, each dried aliquot of the composition comprises an amount of water that is less than 0.01% by weight of the dried aliquot.

For example, the dried aliquots can be stored in a freezer at a temperature between −15° C. and −90° C. The dried aliquots can be stored for a desired time period, such as for at least 24 hours, or for at least one week, or for between one week and one month, or for between one month and six months. Drying is preferably accomplished without destroying a significant portion of the biological activity of the reverse transcriptase or RNA polymerase. For example, drying can be achieved by lyophilization of the aliquots of the liquid composition.

It is also within the scope of the invention to freeze the aliquots of the liquid composition, store the frozen aliquots at a temperature below −15° C. (e.g., between −15° C. and −90° C.) for a period of time (e.g., for at least 24 hours, or for at least one week, or for between one week and one month, or for between one month and six months), and then dry the frozen aliquots to produce dried aliquots of the composition, wherein each dried aliquot of the composition comprises an amount of water that is less than 0.1% by weight of the dried aliquot, and the dried aliquots are stored at a temperature below −15° C. (e.g., for at least 24 hours, or for at least one week, or for between one week and one month, or for between one month and six months).

In the practice of the invention, the frozen aliquots can optionally be thawed and then stored at a temperature between 0° C. and 6° C. for a period of from 4 hours to 12 hours (e.g., stored on ice, or in a refrigerated container maintained at a temperature of between 0° C. and 6° C.) before being used. Similarly, the dried aliquots can optionally be rehydrated and then stored at a temperature between 0° C. and 6° C. for a period of from 4 hours to 12 hours before being used. Thus, numerous liquid reaction mixtures can be stored at a temperature between 0° C. and 6° C. until they are ready to be used.

The dried aliquots of the liquid composition may be rehydrated to produce a rehydrated liquid composition in which the concentrations of the various components are the same as in the liquid composition that was dried to produce the dried aliquots. Alternatively, the concentrations of the various components in the rehydrated liquid composition may be different from the concentrations of the various components in the liquid composition that was dried to produce the dried aliquots.

Compositions of the Invention: In another aspect the present invention provides compositions useful for synthesizing nucleic acid molecules. The compositions of the invention contain little, if any, water, and can be stored at a temperature below −15° C. for an extended period (e.g., for several months) before being dissolved in water and used to synthesize nucleic acid molecules.

A first group of compositions each include: a reverse transcriptase; a Tris buffer; a potassium salt; a magnesium salt; at least two different nucleotides; an oligonucleotide; a reducing agent; and an amount of water that is less than 0.1%, by weight, of the composition. Suitable reverse transcriptases, Tris buffers, potassium salts, magnesium salts, nucleotides, oligonucleotides and reducing agents are described herein in connection with the methods of the invention for storing a composition useful for synthesizing nucleic acid molecules. In some embodiments, the amount of water in a composition of the first group of compositions is less than 0.05%, by weight, of the composition. In some embodiments, the amount of water in a composition of the first group of compositions is less than 0.01%, by weight, of the composition. In some embodiments, there is no detectable water in a composition of the first group of compositions. A composition of the first group of compositions can optionally include an RNase inhibitor. An individual aliquot of a composition of the first group of compositions can include, for example, from 1 unit to 5 units of a reverse transcriptase.

A second group of compositions each include: an RNA polymerase, a Tris buffer, a sodium salt, a magnesium salt, at least two different nucleotides, a reducing agent, and an amount of water that is less than 0.1%, by weight, of the composition. Suitable RNA polymerases, Tris buffers, sodium salts, magnesium salts, nucleotides, and reducing agents are described herein in connection with the methods of the invention for storing a composition useful for synthesizing nucleic acid molecules. In some embodiments, the amount of water in a composition of the second group of compositions is less than 0.05%, by weight, of the composition. In some embodiments, the amount of water in a composition of the second group of compositions is less than 0.01%, by weight, of the composition. In some embodiments, there is no detectable water in a composition of the second group of compositions. A composition of the second group of compositions can optionally include an RNase inhibitor, and/or spermidine, and/or an inorganic pyrophosphatase. An individual aliquot of a composition of the second group of compositions can include, for example, from 10 units to 50 units of an RNA polymerase.

The compositions of the invention are useful for synthesizing nucleic acid molecules when dissolved in water to form a liquid composition. For example, the compositions of the invention that include a reverse transcriptase are useful for synthesizing cDNA molecules complementary to template RNA molecules. Again by way of example, the compositions of the invention that include an RNA polymerase are useful for synthesizing RNA molecules complementary to template DNA molecules (e.g., template cDNA molecules). The synthesized cDNA and RNA molecules are both useful, for example, as probes for hybridizing to immobilized nucleic acid molecules, such as DNA molecules attached to a silicon or glass substrate (e.g., a DNA microchip). Thus, the compositions of the invention are useful, for example, for synthesizing DNA or RNA probes for screening populations of nucleic acid molecules immobilized on a gene chip, or for screening nucleic acid libraries cloned into microorganisms, or for probing DNA or RNA molecules present on a Northern or Southern blot. Such screening may be conducted for a variety of purposes, such as identifying genes that are expressed in a particular living tissue, or for identifying genes that are expressed at an elevated level in a particular living tissue, or for identifying genes that are expressed in response to a biologically active molecule (e.g., a drug molecule) in a particular living tissue.

Containers provided by the invention: In another aspect the present invention provides containers that each include multiple receptacles, wherein at least 50% of the receptacles (e.g., at least 60%, or at least 70%, or at least 80%, or at least 90%, or all of the receptacles) contain a composition selected from the group consisting of (a) a frozen liquid composition comprising from 1000 units/mL to 5000 units/mL of a reverse transcriptase, (b) a frozen liquid composition comprising from 10,000 units/mL to 50,000 units/mL of an RNA polymerase, and (c) a non-liquid composition (e.g., a dried powder) comprising a reverse transcriptase or an RNA polymerase and an amount of water that is less than 0.1%, by weight, of the non-liquid composition.

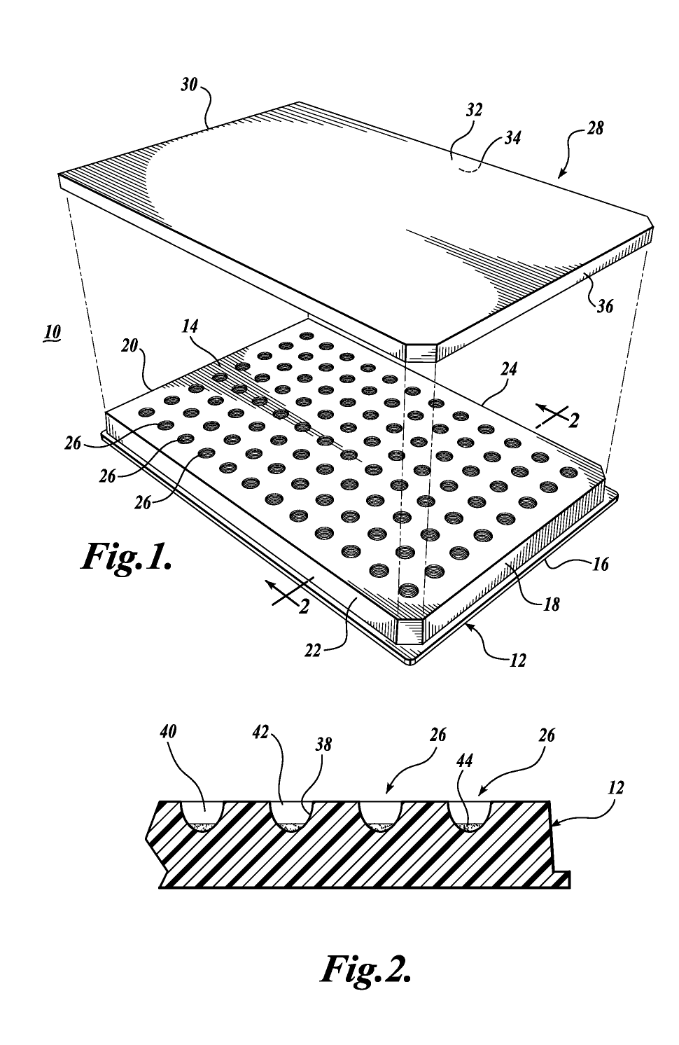

FIG. 1 shows a perspective view of a representative container 10 of the present invention. Container 10 includes a body 12 including an upper surface 14, a lower surface 16 disposed opposite upper surface 14, a first end 18, a second end 20 disposed opposite first end 18, a first face 22, and a second face 24 disposed opposite first face 22. Body 12 defines multiple receptacles 26. A lid 28 may optionally cover upper surface 14. As shown in FIG. 1, lid 28 includes a lid body 30 defining an outer surface 32, an inner surface 34 and a lip 36 that extends around the perimeter of lid body 30.

As shown more clearly in FIG. 2, in the representative embodiment of container 10, each receptacle 26 is generally hemispherical and includes a receptacle wall 38 defining a receptacle lumen 40 that opens onto upper surface 14 of container body 12 through opening 42. Receptacle lumen 40 of at least 50% of receptacles 26 includes a composition 44, wherein composition 44 is selected from the group consisting of (a) a frozen liquid composition comprising from 1000 units/mL to 5000 units/mL of a reverse transcriptase (referred to as composition 44(a)), (b) a frozen liquid composition comprising from 10,000 units/mL to 50,000 units/mL of an RNA polymerase (referred to as composition 44(b)), and (c) a non-liquid composition (e.g., dried powder) comprising a reverse transcriptase or an RNA polymerase and an amount of water that is less than 0.1%, by weight, of the non-liquid composition (referred to as composition 44(c)).

In operation, aliquots of composition 44(a), and/or composition 44(b), and/or a liquid composition which, when dried, yields composition 44(c), are dispensed into lumen 40 of at least 50% of receptacles 26. Different receptacles 26 can contain different compositions 44 (e.g., some receptacles 26 contain composition 44(a), while other receptacles 26 contain composition 44(b). If desired, all receptacles 26 contain the same composition 44 (e.g., all receptacles 26 contain composition 44(a)). Composition 44 is frozen or dried within receptacles 26 (e.g., by placing container 10 within a freezer or lyophilizer). If composition 44(a) or composition 44(b) is/are present in receptacles 26, then container 10 is placed in an environment where the temperature is sufficiently low to freeze composition 44(a) and composition 44(b). For example, container 10 may be placed in a freezer, that maintains an internal temperature lower than −15° C., for a period of time sufficient to freeze composition 44(a) and composition 44(b). Typically, lid 28 is placed over upper surface 14 so that lid inner surface 34 contacts at least a portion of container body upper surface 14. If necessary, a sealing agent (e.g., a layer of plastic film, such as Saran Wrap) may be wrapped around first face 22, second face 24, first end 18 and second end 20 of container body 12, and around lid lip 36, to seal any space that may exist between lid lip 36 and container body 12, thereby preventing the unwanted entry or exit of moisture from receptacles 26. For example, container 10 may be sealed using the ALPS 300™ automated laboratory plate sealer (sold by Abgene, Abgene House, Blenheim Road, Epsom, KT19 9AP, United Kingdom).

In the exemplary embodiment shown in FIG. 1, container 10 has a generally rectangular shape, but container 10 can have any shape, such as square or circular. Similarly, receptacles 26 can have any desired shape provided that they are capable of containing composition 44. Lid 28 is suitably dimensioned to fit over upper surface 14 of container body 12. Container body 12 and lid 28 may be made from any suitable material, or mixtures of suitable materials. Typically, container body 12 and lid 28 are made from plastic.

Examples of commercially-available containers that include wells that can contain an aliquot of composition 44 to yield container 10 of the invention include 96-well plastic plates manufactured by Island Scientific (7869 NE Day Rd West, Bainbridge Island, Wash. 98110), or by MWG Biotech (4170 Mendenhall Oaks Parkway, Suite 160, High Point, N.C. 27265).

Container 10 can include any desired number of receptacles 26. By way of example, container 10 can include from 10 to 50 receptacles 26, or, for example, container 10 can include from 50 to 100 receptacles 26 (e.g., 96 receptacles 26), or, for example, container 10 can include from 100 to 200 receptacles 26, or, for example, container 10 can include more than 200 receptacles 26.

Liquid compositions, comprising from 1000 units/mL to 5000 units/mL of a reverse transcriptase, or comprising from 10,000 units/mL to 50,000 units/mL of an RNA polymerase, that are suitable for inclusion in the containers of the invention include all of the liquid compositions described herein in the section entitled “Liquid compositions that include a reverse transcriptase”, and in the section entitled “Liquid compositions that include an RNA polymerase”.

Non-liquid compositions comprising a reverse transcriptase or an RNA polymerase and an amount of water that is less than 0.1%, by weight, of the non-liquid composition, that are suitable for inclusion in the containers of the invention include all of the first and second groups of compositions described herein in the section entitled “Compositions of the Invention”.

Containers 10 of the present invention are useful, for example, for storing frozen, and/or dehydrated, reaction mixtures useful for synthesizing nucleic acid molecules. Thus, for example, containers 10 of the present invention are useful in the practice of the methods of the invention for storing compositions useful for synthesizing nucleic acid molecules. Thus, for example, multiple aliquots of a liquid composition comprising from 1000 units/mL to 5000 units/mL of a reverse transcriptase, or from 10,000 units/mL to 50,000 units/mL of an RNA polymerase, may be dispensed into receptacles 26 of container 10; and the aliquots may be frozen and stored in container 10 at a temperature below −15° C. (e.g., by placing container 10 in a freezer); or the aliquots may be frozen and dried (e.g., by lyophilization) so that each dried aliquot of the composition comprises an amount of water that is less than 0.1%, by weight, of the dried aliquot, and then stored at a temperature below −15° C.

The following examples merely illustrate the best mode now contemplated for practicing the invention, but should not be construed to limit the invention.

### EXAMPLE 1

This Example compares the yield and fidelity of cRNA synthesis using either reaction mixtures that were prepared and then immediately used, or reaction mixtures that were stored in accordance with the present invention before being used to synthesize nucleic acid molecules. The cRNA was synthesized via a scheme that first used mRNA (in a sample of total RNA) as a template to synthesize double stranded cDNA, then used the cDNA as a template for cRNA synthesis.

cRNA was made from total RNA (including mRNA) from two types of cells: Jurkat cells and K562 cells. Thus, half the samples in each reaction plate were from Jurkat cells, and half the samples in each reaction plate were from K562 cells. 5 μg total RNA was used as starting material for each cRNA synthesis reaction. Total RNA from Jurkat cells and from K562 cells was purchased from Ambion, Inc., 2130 Woodward, Austin Tex. 78744-1832, U.S.A.

Double-Stranded cDNA synthesis using reaction mixtures that were not stored prior to use: An MMLV-RT reaction cocktail was prepared thirty minutes prior to the start of the reverse transcription reaction. The composition of the MMLV-RT reaction cocktail is set forth in Table 1. A 14.5 μl aliquot of the prepared cocktail was dispensed into each well of a 96-well plate. This plate was used as the source plate within 20 minutes of dispensing the aliquots of MMLV-RT reaction cocktail. 9.5 μl aliquots of MMLV-RT reaction cocktail were transferred from the source plate into the MMLV-RT reaction plate. The MMLV-RT enzyme synthesized both first and second strand cDNA in the same reaction mixture.

TABLE 1

| Components | Concentration in Reaction |
|---|---|
| Tris-HCl pH 8.3 | 50 mM |
| KCl | 75 mM |
| MgCl₂ | 3 mM |
| dNTPs | 0.5 mM each |
| RNAguard | 3.2-6.5 units/μL |
| Random Hexamer | 1.0 ng/μL |
| DTT | 10 mM |
| MMLV RT | 2.5 units/μL |

Double-Stranded cDNA synthesis using reaction mixtures that were stored prior to use in accordance with the present invention: MMLV-RT reaction cocktail was prepared. The composition of the MMLV-RT reaction cocktail is set forth in Table 1. 10 μl aliquots of the MMLV-RT reaction cocktail were dispensed into individual wells of a group of 96-well plates. The plates were then sealed and processed under each of the following conditions: Frozen and stored at −20° C. for a period of from 24 hours to one week; frozen and stored at −80° C. for 30 minutes for a period of from one week to one month; and lyophilized and stored at −80° C. for a period of from one month to six months.

After storage under the foregoing conditions, plates of enzyme cocktails were removed from storage and put into the reagent hotel of the Fully Automated System (disclosed in U.S. provisional patent application Ser. No. 60/432,200) at 4° C. The enzyme plates remained in the hotel from 30 minutes to 6 hours before being used to synthesize double stranded cDNA. The enzyme plates were used both as the enzyme source plates and as the reverse transcriptase reaction plates. cRNA synthesis using reaction mixtures that were not stored prior to use: T7 RNA polymerase reaction cocktails were prepared thirty minutes prior to the start of the in vitro transcription (IVT) reaction. The composition of the T7 RNA polymerase reaction cocktail is set forth in Table 2. A 65 μl aliquot of T7 RNA polymerase reaction cocktail was dispensed into each well of a 96-well plate which was used as the source plate within 20 minutes of dispensing the T7 RNA polymerase reaction cocktail. 60 μl aliquots of T7 RNA polymerase reaction cocktail were transferred from the source plate into the plate containing the reverse transcriptase reaction mixture (that was not stored prior to use) and double-stranded cDNA products.

TABLE 2

| Components | Concentration in Reaction |
|---|---|
| Tris-HCl pH 7.5 | 40.0 mM |
| MgCl2 | 14.25 mM |
| NaCl | 10.0 mM |
| Spermidine | 2.0 mM |
| RNAguard | 125-250 units/mL |
| A, C, G | 2.50 mM each |
| U | 1.88 mM |
| aa-UTP | 0.60 mM |
| DTT | 7.5 mM |
| IPPase | 15 units/mL |
| T7 Polymerase | 25,000 units/mL |

cRNA synthesis using reaction mixtures that were stored prior to use: T7 RNA polymerase reaction cocktail was prepared according to Table 2. 60 μl aliquots of the T7 RNA polymerase reaction cocktail were dispensed into individual wells of a group of 96-well plates. The plates were then sealed and processed under each of the following conditions: Frozen and stored at −20° C. for a period of from 24 hours to one week; frozen and stored at −80° C. for 30 minutes for a period of from one week to one month; and lyophilized and stored at −80° C. for a period of from one month to six months.

After storage under the foregoing conditions, plates of enzyme cocktails were removed from storage and put into the reagent hotel of the FAS (disclosed in U.S. provisional patent application Ser. No. 60/432,200) at 4° C. The enzyme plates remained in the hotel from 30 minutes to 6 hours before being used. Reverse transcriptase reaction mixture (including double stranded cDNA products), that had been stored prior to use, was added to the RNA polymerase reaction mixture and cRNA was synthesized.

Table 3 shows the yield of cRNA for each of the foregoing reaction conditions. The abbreviation SOP refers to standard operating procedure. The abbreviation FAS refers to Fully Automated System. cRNA yield is presented as the mean (n=96) with standard deviations. The mean yield of cRNA for the same reaction conditions in two separate plates are shown to demonstrate the reproducibility of the results between plates. The mean yield is for all the reactions in a plate, and does not distinguish between reactions that used total RNA from Jurkat cells as starting material, and reactions that used total RNA from K562 as starting material.

TABLE 3

| Experiment | Enzyme Storage Conditions | cRNA yield (μg) |
|---|---|---|
| SOP | Fresh Enzyme | 48.28 ± 4.76 |
| SOP | Fresh Enzyme | 46.55 ± 1.82 |
| SOP | Stored at −80° C. for 7 days | 45.84 ± 2.74 |
| SOP | Stored at −80° C. for 30 days | 46.40 ± 3.09 |
| FAS | Fresh Enzyme | 48.28 ± 4.76 |
| FAS | Fresh Enzyme | 46.14 ± 4.06 |
| FAS | Stored at −80° C. for 7 days | 45.17 ± 3.00 |
| FAS | Stored at −80° C. for 7 days | 45.77 ± 2.25 |
| FAS | Stored at −80° C. for 30 days | 45.79 ± 5.99 |
| FAS | Stored at −80° C. for 30 days | 48.85 ± 4.13 |

As shown in Table 3, the cRNA yields from the reactions using the fresh enzyme cocktails are similar to the cRNA yields from the reactions using the frozen enzyme cocktails.

The method described in this Example was used to make cRNA from K562 cells and Jurkat cells. The cRNA was used to detect differential gene expression between K562 cells and Jurkat cells by probing microchips bearing several hundred different oligonucleotides that specifically hybridize to several hundred different genes (or RNA molecules derived therefrom). The sensitivity and specificity of the cRNA probes produced using stored reaction mixtures was not significantly different from the sensitivity and specificity of the cRNA probes produced using reaction mixtures that were not stored.

### EXAMPLE 2

This Example compares the yield and fidelity of first strand cDNA synthesis using reverse transcriptase reaction mixtures that were prepared and then immediately used to synthesize first strand cDNA, versus the yield and fidelity of first strand cDNA synthesis using identical reverse transcriptase reaction mixtures that were stored in accordance with the present invention before being used to synthesize first strand cDNA. 3 μg cRNA, prepared as described in Example 1, was the starting material for each first strand cDNA synthesis reaction.

First strand cDNA synthesis using reaction mixtures that were not stored prior to use: Reaction cocktail containing SuperScript™ II RNase H⁻ reverse transcriptase (Invitrogen Corp., Carlsbad, Calif.) was prepared thirty minutes prior to the start of the reverse transcription reaction. The composition of the Superscript II reaction cocktail is set forth in Table 4. A 75 μl aliquot of the prepared cocktail was dispensed into each well of a 96-well plate. This plate was used as the source plate within 20 minutes of dispensing the aliquots of Superscript II reaction cocktail. 35 μl aliquots of Superscript II reaction cocktail were transferred from the source plate into the Supercript II reaction plate.

TABLE 4

| Components | Concentration in Reaction |
|---|---|
| Tris-HCl pH 8.3 | 71 mM |
| KCl | 107 mM |
| MgCl₂ | 11 mM |
| dNTPs | 0.7 mM each |
| amino allyl-UTP | 0.7 mM |
| DTT | 14 mM |
| SuperScript ™ II | 14 units/μL |

First strand cDNA synthesis using reaction mixtures that were stored prior to use in accordance with the present invention: Superscript™ II reaction cocktail was prepared. The composition of the Superscript™ II reaction cocktail is set forth in Table 4. A 35 μl aliquot of the Superscript™ II reaction cocktail was dispensed into each well of a 96-well plate. The plate was then sealed and stored under each of the following conditions: Frozen and stored at −20° C. for 24 hours to one week; frozen and stored at −80° C. for one week to one month; and lyophilized and stored at −80° C. for one month to six months. After storage under the foregoing conditions, plates of enzyme cocktails were thawed and stored on ice before being used to synthesize cDNA.

As shown in Table 5, cDNA yields are similar when the syntheses are carried out with the fresh enzyme cocktails or the frozen enzyme cocktails. The cDNA yield values shown in Table 5 are each the average of sixteen measurements.

Additionally, hybridization of the cDNA produced using stored reaction mixtures revealed an almost identical gene expression pattern as hybridization of the cDNA produced using reaction mixtures that had not been stored prior to use.

TABLE 5

| Experiment | Enzyme Storage | cDNA yield (μg) |
|---|---|---|
| K562 | Fresh Enzyme | 2.27 ± 0.41 |
| K562 | Stored at −80° C. for 7 days | 2.52 ± 0.23 |
| Jurkat | Fresh Enzyme | 2.38 ± 0.27 |
| Jurkat | Stored at −80° C. for 7 days | 2.41 ± 0.15 |

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for storing a composition useful for synthesizing nucleic acid molecules, wherein the method comprises the steps of:
(a) freezing multiple aliquots of a liquid composition, wherein each aliquot comprises from 1000 units/mL to 5000 units/mL of a reverse transcriptase, or from 10,000 units/mL to 50,000 units/mL of an RNA polymerase, wherein the multiple aliquots of the liquid composition are disposed within multiple receptacles defined by a container body; and
(b) a step selected from the group consisting of (1) storing the frozen aliquots at a temperature below −15° C., and (2) drying the frozen aliquots to produce dried aliquots of the composition, wherein each dried aliquot of the composition comprises an amount of water that is less than 0.1% by weight of the dried aliquot, and storing the dried aliquots at a temperature below −15° C.

2. The method of claim 1 wherein from 10 to 100 aliquots of the liquid composition are disposed within from 10 to 100 receptacles.

3. The method of claim 2 wherein the container body consists essentially of plastic.

4. The method of claim 3 wherein the container body defines 96 receptacles, and 96 aliquots of the liquid composition are disposed within the 96 receptacles.

5. The method of claim 1 wherein the frozen aliquots are stored at a temperature below −15° C.

6. The method of claim 5 wherein the frozen aliquots are stored at a temperature between −15° C. and −90° C.

7. The method of claim 5 wherein the frozen aliquots are stored at a temperature between −15° C. and −90° C. for at least one day.

8. The method of claim 5 wherein the frozen aliquots are stored at a temperature between −15° C. and −90° C. for at least one week.

9. The method of claim 5 wherein the frozen aliquots are stored at a temperature between −15° C. and −90° C. for between one week and one month.

10. The method of claim 1 wherein the frozen aliquots are dried to produce dried aliquots of the composition, wherein each dried aliquot of the composition comprises an amount of water that is less than 0.1% by weight of the dried aliquot, and the dried aliquots are stored at a temperature below −15° C.

11. The method of claim 10 wherein each dried aliquot of the composition comprises an amount of water that is less than 0.05% by weight of the dried aliquot.

12. The method of claim 10 wherein the frozen aliquots are dried by lyophilization.

13. The method of claim 10 wherein the dried aliquots are stored at a temperature between −15° C. and −90° C.

14. The method of claim 10 wherein the dried aliquots are stored at a temperature between −15° C. and −90° C. for at least 24 hours.

15. The method of claim 10 wherein the dried aliquots are stored at a temperature between −15° C. and −90° C. for at least one week.

16. The method of claim 10 wherein the dried aliquots are stored at a temperature between −15° C. and −90° C. for between one week and one month.

17. The method of claim 1 wherein the liquid composition comprises from 1000 units/mL to 5000 units/mL of a reverse transcriptase.

18. The method of claim 17 wherein the reverse transcriptase is an RNase H⁻ reverse transcriptase.

19. The method of claim 17 wherein the reverse transcriptase is an RNase H⁺ reverse transcriptase.

20. The method of claim 17 wherein the reverse transcriptase is a Moloney murine leukemia virus reverse transcriptase.

21. The method of claim 17 wherein each aliquot of the liquid composition comprises a Tris buffer, a potassium salt, a magnesium salt, at least two different nucleotides, an oligonucleotide, and a reducing agent.

22. The method of claim 21 wherein the potassium salt is potassium chloride.

23. The method of claim 21 wherein the magnesium salt is magnesium chloride.

24. The method of claim 21 wherein the liquid composition comprises at least four members of the group consisting of adenine, cytosine, guanine, thymidine, and a derivative of any of the foregoing nucleotides.

25. The method of claim 21 wherein the liquid composition comprises adenine, cytosine, guanine, and thymidine.

26. The method of claim 21 wherein the oligonucleotide is a DNA molecule.

27. The method of claim 26 wherein the DNA molecule consists of from 9 to 50 nucleotides.

28. The method of claim 21 wherein the reducing agent is dithiothreitol.

29. The method of claim 21 wherein:
(a) the reverse transcriptase is present in the liquid composition at a concentration in the range of from 1000 units/mL to 5000 units/mL;
(b) the Tris buffer is present in the liquid composition at a concentration in the range of from 10 mM to 100 mM;
(c) the potassium salt is present in the liquid composition at a concentration in the range of from 10 mM to 100 mM;
(d) the magnesium salt is present in the liquid composition at a concentration in the range of from 1.0 mM to 10 mM;
(e) the nucleotides are each present in the liquid composition at a concentration in the range of from 0.5 mM to 5 mM;
(f) the oligonucleotide is present in the liquid composition at a concentration in the range of from 0.01 mM to 0.05 mM; and
(g) the reducing agent is present in the liquid composition at a concentration in the range of from 2.0 mM to 20 mM.

30. The method of claim 21 wherein
(a) the reverse transcriptase is present in the liquid composition at a concentration of 2.5 units/μL;
(b) the Tris buffer is present in the liquid composition at a concentration of 50 mM;
(c) the potassium salt is potassium chloride that is present in the liquid composition at a concentration of 75 mM;
(d) the magnesium salt is magnesium chloride that is present in the liquid composition at a concentration of 3 mM;
(e) the nucleotides are each present in the liquid composition at a concentration of 0.5 mM;
(f) the oligonucleotide is present in the liquid composition at a concentration of 1.0 ng/μL; and
(g) the reducing agent is dithiothreitol that is present in the liquid composition at a concentration of 10 mM.

31. The method of claim 21 wherein the liquid composition further comprises an RNase inhibitor.

32. The method of claim 31 wherein the RNase inhibitor is present in the liquid composition at a concentration in the range of from 0.1 units/μL to 1.0 units/μL.

33. The method of claim 21 wherein the RNase inhibitor is RNAguard that is present in the liquid composition at a concentration of 3.2 units/μL to 6.5 units/μL.

34. The method of claim 1 wherein the liquid composition comprises from 10,000 units/mL to 50,000 units/mL of an RNA polymerase.

35. The method of claim 34 wherein the RNA polymerase is a T7 RNA polymerase.

36. The method of claim 34 wherein each aliquot of the liquid composition comprises a Tris buffer, a sodium salt, a magnesium salt, at least two different nucleotides, and a reducing agent.

37. The method of claim 36 wherein the sodium salt is sodium chloride.

38. The method of claim 36 wherein the magnesium salt is magnesium chloride.

39. The method of claim 36 wherein the liquid composition comprises at least four members of the group consisting of adenine, cytosine, guanine, uracil, and a derivative of any of the foregoing nucleotides.

40. The method of claim 36 wherein the liquid composition comprises adenine, cytosine, guanine, uracil and an amino allyl uracil nucleotide.

41. The method of claim 36 wherein the reducing agent is dithiothreitol.

42. The method of claim 36 wherein:
(a) the RNA polymerase is present in the liquid composition at a concentration in the range of from 10 units/μL to 50 units/μL;
(b) the Tris buffer is present in the liquid composition at a concentration in the range of from 10 mM to 100 mM;
(c) the sodium salt is present in the liquid composition at a concentration in the range of from 2.0 mM to 20 mM;
(d) the magnesium salt is present in the liquid composition at a concentration in the range of from 1.0 mM to 15 mM;
(e) adenine, cytosine and guanine are each present in the liquid composition at a concentration in the range of from 0.5 mM to 5.0 mM;
(f) uracil is present in the liquid composition at a concentration in the range of from 0.2 mM to 2.0 mM;
(g) an amino allyl uracil nucleotide is present in the liquid composition at a concentration in the range of from 0.05 mM to 1.0 mM; and
(h) the reducing agent is present in the liquid composition at a concentration in the range of from 2.0 mM to 20 mM.

43. The method of claim 36 wherein:
(a) the RNA polymerase is present in the liquid composition at a concentration of 25,000 units/mL;
(b) the Tris buffer is present in the liquid composition at a concentration of 40 mM;
(c) sodium chloride is present in the liquid composition at a concentration of 10 mM;
(d) magnesium chloride is present in the liquid composition at a concentration of 14.25 mM;
(e) adenine, cytosine and guanine are each present in the liquid composition at a concentration of 2.5 mM;
(f) uracil is present in the liquid composition at a concentration of 1.88 mM;
(g) an amino allyl uracil derivative is present in the liquid composition at a concentration of 0.6 mM; and
(h) dithiothreitol is present in the liquid composition at a concentration of 7.5 mM.

44. The method of claim 36 wherein the liquid composition further comprises spermidine.

45. The method of claim 44 wherein the spermidine is present in the liquid composition at a concentration in the range of from 1.0 mM to 5 mM.

46. The method of claim 36 wherein the liquid composition further comprises an RNase inhibitor.

47. The method of claim 46 wherein the RNase inhibitor is present in the liquid composition at a concentration in the range of from 100 U/mL to 500 U/mL.

48. The method of claim 36 wherein the liquid composition further comprises an IPPase.

49. The method of claim 48 wherein the IPPase is present in the liquid composition at a concentration in the range of from 5 units/mL to 50 units/mL.

50. The method of claim 1 wherein the frozen aliquots are stored at a temperature below −15° C., then thawed and stored at a temperature between 0° C. and 6° C. for a period of from 4 hours to 12 hours.

51. The method of claim 1 wherein the dried aliquots are stored at a temperature below −15° C., then rehydrated and stored at a temperature between 0° C. and 6° C. for a period of from 4 hours to 12 hours.

52. A container comprising multiple receptacles, wherein at least 50% of the receptacles contain a composition selected from the group consisting of (a) a frozen liquid composition comprising from 1000 units/mL to 5000 units/mL of a reverse transcriptase, (b) a frozen liquid composition comprising from 10,000 units/mL to 50,000 units/mL of an RNA polymerase, and (c) a non-liquid composition comprising a reverse transcriptase or an RNA polymerase and an amount of water that is less than 0.1%, by weight, of the non-liquid composition.

53. The container of claim 52 wherein at least 70% of the receptacles contain a composition selected from the group consisting of (a) a frozen liquid composition comprising from 1000 units/mL to 5000 units/mL of a reverse transcriptase, (b) a frozen liquid composition comprising from 10,000 units/mL to 50,000 units/mL of an RNA polymerase, and (c) a non-liquid composition comprising a reverse transcriptase or an RNA polymerase and an amount of water that is less than 0.1%, by weight, of the non-liquid composition.

54. The container of claim 52 wherein at least 90% of the receptacles contain a composition selected from the group consisting of (a) a frozen liquid composition comprising from 1000 units/mL to 5000 units/mL of a reverse transcriptase, (b) a frozen liquid composition comprising from 10,000 units/mL to 50,000 units/mL of an RNA polymerase, and (c) a non-liquid composition comprising a reverse transcriptase or an RNA polymerase and an amount of water that is less than 0.1%, by weight, of the non-liquid composition.

55. The container of claim 52 wherein all of the receptacles contain a composition selected from the group consisting of (a) a frozen liquid composition comprising from 1000 units/mL to 5000 units/mL of a reverse transcriptase, (b) a frozen liquid composition comprising from 10,000 units/mL to 50,000 units/mL of an RNA polymerase, and (c) a non-liquid composition comprising a reverse transcriptase or an RNA polymerase and an amount of water that is less than 0.1%, by weight, of the non-liquid composition.

56. The container of claim 52 wherein at least 50% of the receptacles contain a frozen liquid composition comprising from 1000 units/mL to 5000 units/mL of a reverse transcriptase.

57. The container of claim 52 wherein at least 50% of the receptacles contain a frozen liquid composition comprising from 10,000 units/mL to 50,000 units/mL of an RNA polymerase.

58. The container of claim 52 wherein at least 50% of the receptacles contain a non-liquid composition comprising a reverse transcriptase or an RNA polymerase and an amount of water that is less than 0.1%, by weight, of the non-liquid composition.

59. The container of claim 52 wherein from 10 to 100 aliquots of the composition are disposed within from 10 to 100 receptacles.

60. The container of claim 52 wherein the container body consists essentially of plastic.

61. The container of claim 52 wherein the container body defines 96 receptacles, and an aliquot of the composition is present within all of the receptacles.

62. The container of claim 52 wherein the liquid composition comprises a Tris buffer, a potassium salt, a magnesium salt, at least two different nucleotides, an oligonucleotide, a reducing agent, from 1000 units/mL to 5000 units/mL of a reverse transcriptase.

63. The container of claim 52 wherein each aliquot of the liquid composition comprises a Tris buffer, a sodium salt, a magnesium salt, at least two different nucleotides, a reducing agent, and an RNA polymerase.

64. A composition useful for synthesizing nucleic acid molecules, said composition comprising:
(a) a reverse transcriptase;
(b) a Tris buffer;
(c) a potassium salt;
(d) a magnesium salt;
(e) two different nucleotides;
(f) an oligonucleotide;
(g) a reducing agent; and
(h) an amount of water that is less than 0.1%, by weight, of the composition.

65. The composition of claim 64 comprising from 1 unit to 5 units of the reverse transcriptase.

66. The composition of claim 64 further comprising an RNase inhibitor.

67. A composition useful for synthesizing nucleic acid molecules, said composition comprising:
(a) an RNA polymerase;
(b) a Tris buffer;
(c) a sodium salt;
(d) a magnesium salt;
(e) two different nucleotides;
(f) a reducing agent; and
(g) an amount of water that is less than 0.1%, by weight, of the composition.

68. The composition of claim 67 comprising from 10 units to 50 units of the RNA polymerase.

69. The composition of claim 67 further comprising an RNase inhibitor.

70. The composition of claim 67 further comprising spermidine.

71. The composition of claim 67 further comprising an inorganic pyrophosphatase.
